(19) United States
(12) Patent Application Publication
(10) Pub. No.: US 2023/0073059 A1
(43) Pub. Date: Mar. 9, 2023

(54) LENSES HAVING DIFFRACTIVE PROFILES WITH ELEVATED SURFACE ROUGHNESS

(71) Applicant: AMO Groningen B.V., Groningen (NL)

(72) Inventors: Hendrik A. Weeber, Groningen (NL); Aixa Alarcon Heredia, Groningen (NL); Carmen Canovas Vidal, Groningen (NL); Franck Gounou, Groningen (NL); Patricia A. Piers, Groningen (NL); Miguel Faria Ribeiro, BRAGA (PT)

(73) Assignee: AMO Groningen B.V., GRONINGEN (NL)

(21) Appl. No.: 17/930,400

(22) Filed: Sep. 7, 2022

Related U.S. Application Data
(60) Provisional application No. 63/261,011, filed on Sep. 8, 2021.

(51) Int. Cl. A61F 2/16

(52) U.S. Cl. CPC A61F 2/1656; A61F 2240/001; A61F 2002/0081

(57) ABSTRACT

Apparatuses, systems and methods for providing improved ophthalmic lenses, particularly intraocular lenses (IOLs), include features for reducing adverse optical effects from diffractive profiles of such a lens. Exemplary ophthalmic lenses can include an optic including a diffractive profile including a transition zone having an elevated surface roughness.

### CROSS-REFERENCE TO RELATED APPLICATIONS

This application claims priority to U.S. Provisional Patent Application No. 63/261,011, filed Sep. 8, 2021, which is incorporated herein by reference in its entirety.

### BACKGROUND

Embodiments of the present disclosure relate to vision treatment techniques and in particular, to ophthalmic lenses such as, for example, contact lenses, corneal inlays or onlays, or intraocular lenses (IOLs) including, for example, phakic IOLs and piggyback IOLs (i.e. IOLs implanted in an eye already having an IOL).

Presbyopia is a condition that affects the accommodation properties of the eye. As objects move closer to a young, properly functioning eye, the effects of ciliary muscle contraction and zonular relaxation allow the lens of the eye to change shape, and thus increase its optical power and ability to focus at near distances. This accommodation can allow the eye to focus and refocus between near and far objects.

Presbyopia normally develops as a person ages and is associated with a natural progressive loss of accommodation. The presbyopic eye often loses the ability to rapidly and easily refocus on objects at varying distances. The effects of presbyopia usually become noticeable after the age of 45 years. By the age of 65 years, the crystalline lens has often lost almost all elastic properties and has only a limited ability to change shape.

Along with reductions in accommodation of the eye, age may also induce clouding of the lens due to the formation of a cataract. A cataract may form in the hard central nucleus of the lens, in the softer peripheral cortical portion of the lens, or at the back of the lens. Cataracts can be treated by the replacement of the cloudy natural lens with an artificial lens. An artificial lens replaces the natural lens in the eye, with the artificial lens often being referred to as an intraocular lens or “IOL.”

Monofocal IOLs are intended to provide vision correction at one distance only, usually the far focus. At the very least, since a monofocal IOL provides vision treatment at only one distance and since the typical correction is for far distance, spectacles are usually needed for good vision at near distances and sometimes for good vision at intermediate distances. The term “near vision” generally corresponds to vision provided when objects are at a distance from the subject eye at equal; or less than 1.5 feet. The term “distant vision” generally corresponds to vision provided when objects are at a distance of at least about 5-6 feet or greater. The term “intermediate vision” corresponds to vision provided when objects are at a distance of about 1.5 feet to about 5-6 feet from the subject eye. Such characterizations of near, intermediate, and far vision correspond to those addressed in Morlock R, Wirth R J, Tally S R, Garufis C, Heichel C W D, Patient-Reported Spectacle Independence Questionnaire (PRSIQ): Development and Validation. Am J Ophthalmology 2017; 178:101-114.

There have been various attempts to address limitations associated with monofocal IOLs. For example, multifocal IOLs have been proposed that deliver, in principle, two foci, one near and one far, optionally with some degree of intermediate focus. Such multifocal, or bifocal, IOLs are intended to provide good vision at two distances, and include both refractive and diffractive multifocal IOLs. In some instances, a multifocal IOL intended to correct vision at two distances may provide a near (add) power of about 3.0 or 4.0 diopters.

Multifocal IOLs may, for example, rely on a diffractive optical surface to direct portions of the light energy toward differing focal distances, thereby allowing the patient to clearly see both near and far objects. Multifocal ophthalmic lenses (including contact lenses or the like) have also been proposed for treatment of presbyopia without removal of the natural crystalline lens. Diffractive optical surfaces, either monofocal or multifocal, may also be configured to provide reduced chromatic aberration.

Diffractive monofocal and multifocal lenses can make use of a material having a given refractive index and a surface curvature which provide a refractive power. Diffractive lenses have a diffractive profile which confers the lens with a diffractive power that contributes to the overall optical power of the lens. The diffractive profile is typically characterized by a number of diffractive zones. When used for ophthalmic lenses these zones are typically annular lens zones, or echelettes, spaced about the optical axis of the lens. Each echelette may be defined by an optical zone, a transition zone between the optical zone and an optical zone of an adjacent echelette, and an echelette geometry. The echelette geometry includes an inner and outer diameter and a shape or slope of the optical zone, a height or step height, and a shape of the transition zone. The surface area or diameter of the echelettes largely determines the diffractive power(s) of the lens and the step height of the transition between echelettes largely determines the light distribution between the different powers. Together, these echelettes form a diffractive profile.

A multifocal diffractive profile of the lens may be used to mitigate presbyopia by providing two or more optical powers; for example, one for near vision and one for far vision. The lenses may also take the form of an intraocular lens placed within the capsular bag of the eye, replacing the original lens, or placed in front of the natural crystalline lens. The lenses may also be in the form of a contact lens, most commonly a bifocal contact lens, or in any other form mentioned herein.

Although multifocal ophthalmic lenses lead to improved quality of vision for many patients, additional improvements would be beneficial. For example, some pseudophakic patients experience undesirable visual effects (dysphotopsia), e.g. glare or halos. Halos may arise when light from the unused focal image creates an out-of-focus image that is superimposed on the used focal image. For example, if light from a distant point source is imaged onto the retina by the distant focus of a bifocal IOL, the near focus of the IOL will simultaneously superimpose a defocused image on top of the image formed by the distant focus. This defocused image may manifest itself in the form of a ring of light surrounding the in-focus image, and is referred to as a halo. Another area of improvement revolves around the typical bifocality of multifocal lenses. While multifocal ophthalmic lenses typically provide adequate near and far vision, intermediate vision may be compromised.

A lens with an extended range of vision may thus provide certain patients the benefits of good vision at a range of distances, while having reduced or no dysphotopsia. Various techniques for extending the depth of focus of an IOL have been proposed. One technique is embodied in the Tecnis Symfony® lens offered by Johnson & Johnson Vision. One technique may include a bulls-eye refractive principle, and may involve a central zone with a slightly increased power. One technique may include an asphere or include refractive zones with different refractive zonal powers.

Although certain proposed treatments may provide some benefit to patients in need thereof, further advances would be desirable. For example, it would be desirable to provide improved IOL systems and methods that confer enhanced image quality across a wide and extended range of foci without dysphotopsia. Further, improved IOL systems and methods to reduce visual symptoms produced by transition zones of diffractive profiles, such as halo, glare, and scatter may be desired. Embodiments of the present disclosure may provide solutions that address the problems described above, and hence may provide answers to at least some of these outstanding needs.

### BRIEF SUMMARY

Embodiments herein described include ophthalmic lenses including an optic. The optic may include a diffractive profile including at least one echelette having an optical zone and a transition zone, with at least a portion of the transition zone having an elevated surface roughness. The elevated surface roughness may also have a frosting on the transition zone.

The elevated surface roughness may be formed by a tool applied to the transition zone. The tool may comprise a lathe. The tool may form the transition zone. The tool may also form the optical zone. It is also envisioned that the elevated surface roughness may be formed by a mold that the optic is formed in. At least one area of the surface may be polished to reduce the elevated surface roughness of that area, which may include the optical zone of at least one echelette.

The elevated surface roughness may be configured to scatter light striking the transition zone, including adding a textured pattern on at least a portion of the transition zone. The elevated surface roughness may have a greater roughness than a surface of the optical zone. It is further envisioned that the optical zone may have a surface that is optically smooth.

An entirety of the transition zone may have the elevated surface roughness. In addition, the elevated surface roughness may extend to at least a portion of the optical zone. The elevated surface roughness may also have a greater roughness than a surface of at least one optical zone of the plurality of echelettes.

The diffractive profile is made up of a plurality of echelettes, each echelette having an optical zone and a transition zone. At least one of the transition zones of the plurality of echelettes may lack an elevated surface roughness. At least one of the optical zones of the plurality of echelettes may have an elevated surface roughness. In addition, the elevated surface roughness of at least a portion of the transition zone may extend to one or both of at least a portion of an optical zone of at least one echelette or at least a portion of an optical zone of an adjacent echelette.

Embodiments herein described include a method comprising fabricating an optic for an ophthalmic lens, the optic including a diffractive profile including at least one echelette having an optical zone and a transition zone, with at least a portion of the transition zone having an elevated surface roughness. The elevated surface roughness may be configured to scatter light striking the transition zone.

The method may include receiving an ophthalmic lens prescription, and then fabricating the optic based on the ophthalmic lens prescription. Determination of one or more of the diffractive profile or a refractive profile of the optic may be based on the ophthalmic lens prescription.

The method may include forming the elevated surface roughness by applying a tool to the portion of the transition zone to cut the elevated surface roughness into the portion. The tool may comprise of a lathe. It is also envisioned that the elevated surface roughness may be formed by a mold that the optic is formed in.

The method may include forming a surface of the optic having the elevated surface roughness and polishing at least one area of the surface to reduce the elevated surface roughness of the at least one area. A portion of the transition zone may be covered during the polishing.

The method may include fabricating an elevated surface roughness that has a greater roughness than a surface of the optical zone. It is also envisioned that the method may include selectively forming the elevated surface roughness on one or more of the optical zones or transition zones of the plurality of echelettes. The method may include selectively forming the elevated surface roughness on a first one of the transition zones, and selectively forming an optically smooth second one of the transition zones. The method may further comprise selectively forming the elevated surface roughness on a first one of the optical zones, and selectively forming an optically smooth second one of the optical zones. It is further envisioned that the method may include fabricating an elevated surface roughness of at least a portion of a transition zone that extends to one or both of at least a portion of an optical zone of at least one echelette or at least a portion of an optical zone of an adjacent echelette.

Embodiments herein described include a system for fabricating an ophthalmic lens. The system may include a processor configured to determine a diffractive profile of an optic, the optic including a diffractive profile including at least one echelette having an optical zone and a transition zone, with at least a portion of the transition zone having an elevated surface roughness. The elevated surface roughness may be configured to scatter light striking the transition zone. The system may include a manufacturing assembly that fabricates the optic based on the diffractive profile.

The system may further include an input for receiving an ophthalmic lens prescription, wherein the processor is configured to determine one or more of the diffractive profile or a refractive profile of the optic based on the ophthalmic lens prescription.

The manufacturing assembly may be configured to form the elevated surface roughness by applying a tool to the portion of the transition zone to cut the elevated surface roughness into the portion. The tool may comprise a lathe. A system is also envisioned wherein the elevated surface roughness is formed by a mold that the optic is formed in.

The manufacturing assembly may be configured to form a surface of the optic having the elevated surface roughness and polishing at least one area of the surface to reduce the elevated surface roughness of the at least one area. The elevated surface roughness may have a greater roughness than a surface of the optical zone.

The diffractive profile may include a plurality of echelettes, each echelette having an optical zone and a transition zone, and the manufacturing assembly may be configured to selectively form the elevated surface roughness on one or more of the optical zones or transition zones of the plurality of echelettes. The elevated surface roughness of at least a portion of a transition zone may extend to one or both of at least a portion of an optical zone of at least one echelette or at least a portion of an optical zone of an adjacent echelette.

### BRIEF DESCRIPTION OF THE DRAWINGS

FIG. 1A illustrates a cross-sectional view of an eye with an implanted multifocal refractive intraocular lens.

FIG. 1B illustrates a cross-sectional view of an eye having an implanted multifocal diffractive intraocular lens.

FIG. 2A illustrates a front view of a diffractive multifocal intraocular lens.

FIG. 2B illustrates a cross-sectional view of a diffractive multifocal intraocular lens.

FIGS. 3A-3B are graphical representations of a portion of the diffractive profile of a conventional diffractive multifocal lens.

FIG. 4 illustrates a graphical representation of a portion of a diffractive profile according to an embodiment of the present disclosure.

FIG. 5 illustrates a graphical representation of a portion of a diffractive profile according to an embodiment of the present disclosure.

FIG. 6 illustrates a graphical representation of a portion of a diffractive profile according to an embodiment of the present disclosure.

FIG. 7 illustrates a graphical representation of a portion of a diffractive profile according to an embodiment of the present disclosure.

FIG. 8 illustrates an embodiment of a system.

### DETAILED DESCRIPTION

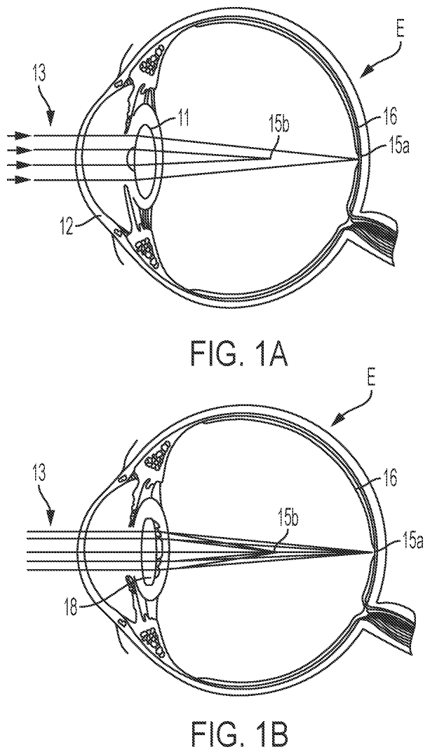

FIGS. 1A, 1B, 2A, 2B, 3A and 3B illustrate multifocal IOL lens geometries, aspects of which are described in U.S. Patent Publication No. 2011-0149236 A1, which is hereby incorporated by reference in its entirety.

FIG. 1A is a cross-sectional view of an eye E fit with a multifocal IOL 11. As shown, multifocal IOL 11 may, for example, comprise a bifocal IOL. Multifocal IOL 11 receives light from at least a portion of cornea 12 at the front of eye E and is generally centered about the optical axis of eye E. For ease of reference and clarity, FIGS. 1A and 1B do not disclose the refractive properties of other parts of the eye, such as the corneal surfaces. Only the refractive and/or diffractive properties of the multifocal IOL 11 are illustrated.

Each major face of lens 11, including the anterior (front) surface and posterior (back) surface, generally has a refractive profile, e.g. biconvex, plano-convex, plano-concave, meniscus, etc. The two surfaces together, in relation to the properties of the surrounding aqueous humor, cornea, and other optical components of the overall optical system, define the effects of the lens 11 on the imaging performance by eye E. Conventional, monofocal IOLs have a refractive power based on the refractive index of the material from which the lens is made, and also on the curvature or shape of the front and rear surfaces or faces of the lens. One or more support elements may be configured to secure the lens 11 to a patient's eye.

Multifocal lenses may optionally also make special use of the refractive properties of the lens. Such lenses generally include different powers in different regions of the lens so as to mitigate the effects of presbyopia. For example, as shown in FIG. 1A, a perimeter region of refractive multifocal lens 11 may have a power which is suitable for viewing at far viewing distances. The same refractive multifocal lens 11 may also include an inner region having a higher surface curvature and a generally higher overall power (sometimes referred to as a positive add power) suitable for viewing at near distances.

Rather than relying entirely on the refractive properties of the lens, multifocal diffractive IOLs or contact lenses can also have a diffractive power, as illustrated by the IOL 18 shown in FIG. 1B. The diffractive power can, for example, comprise positive or negative power, and that diffractive power may be a significant (or even the primary) contributor to the overall optical power of the lens. The diffractive power is conferred by a plurality of concentric diffractive zones which form a diffractive profile. The diffractive profile may either be imposed on the anterior face or posterior face or both.

The diffractive profile of a diffractive multifocal lens directs incoming light into a number of diffraction orders. As light 13 enters from the front of the eye, the multifocal lens 18 directs light 13 to form a far field focus 15a on retina 16 for viewing distant objects and a near field focus 15b for viewing objects close to the eye. Depending on the distance from the source of light 13, the focus on retina 16 may be the near field focus 15b instead. Typically, far field focus 15a is associated with 0^{th} diffractive order and near field focus 15b is associated with the 1^{st} diffractive order, although other orders may be used as well.

Bifocal ophthalmic lens 18 typically distributes the majority of light energy into two viewing orders, often with the goal of splitting imaging light energy about evenly (50%:50%), one viewing order corresponding to far vision and one viewing order corresponding to near vision, although typically, some fraction goes to non-viewing orders.

Corrective optics may be provided by phakic IOLs, which can be used to treat patients while leaving the natural lens in place. Phakic IOLs may be angle supported, iris supported, or sulcus supported. The phakic IOL can be placed over the natural crystalline lens or piggy-backed over another IOL. It is also envisioned that the present disclosure may be applied to inlays, onlays, accommodating IOLs, pseudophakic IOLs, other forms of intraocular implants, spectacles, and even laser vision correction.

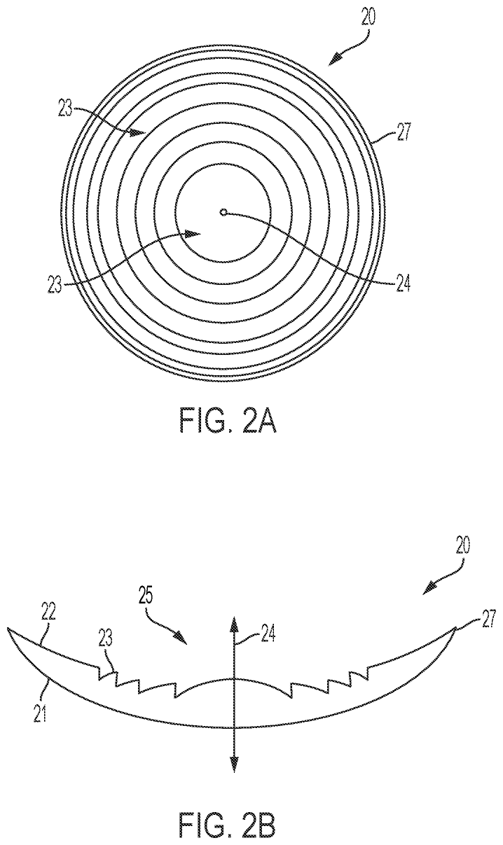

FIGS. 2A and 2B show aspects of a conventional diffractive multifocal lens 20. Multifocal lens 20 may have certain optical properties that are generally similar to those of multifocal IOLs 11, 18 described above. Multifocal lens 20 has an anterior lens face 21 and a posterior lens face 22 disposed about an optical axis 24. The faces 21, 22, or optical surfaces, extend radially outward from the optical axis 24 to an outer periphery 27 of the optic. The faces 21, 22, or optical surfaces, face opposite each other.

When fitted onto the eye of a subject or patient, the optical axis of lens 20 is generally aligned with the optical axis of eye E. The curvature of lens 20 gives lens 20 an anterior refractive profile and a posterior refractive profile. Although a diffractive profile may also be imposed on either anterior face 21 and posterior face 22 or both, FIG. 2B shows posterior face 22 with a diffractive profile. The diffractive profile may extend outward from a central portion 25 of the lens 20. The diffractive profile is characterized by a plurality of annular diffractive zones or echelettes 23 spaced about optical axis 24. While analytical optics theory generally assumes an infinite number of echelettes, a standard multifocal diffractive IOL typically has at least 9 echelettes, and may have over 30 echelettes. For the sake of clarity, FIG. 2B shows only 4 echelettes. Typically, an IOL is biconvex, or possibly plano-convex, or convex-concave, although an IOL could be plano-plano, or other refractive surface combinations.

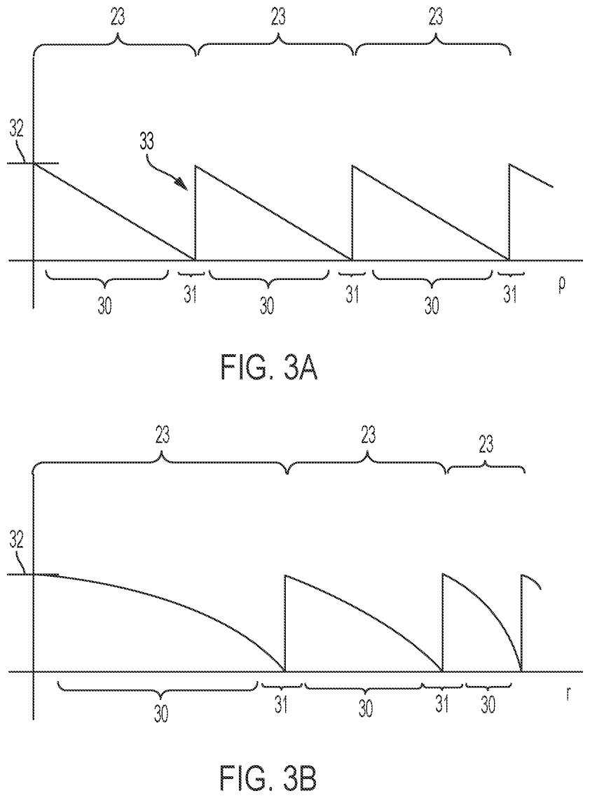

FIGS. 3A and 3B are graphical representations of a portion of a typical diffractive profile of a multifocal lens. While the graph shows only 3 echelettes, typical diffractive lenses extend to at least 9 echelettes to over 32 echelettes. In FIG. 3A, the height 32 of the surface relief profile (from a plane perpendicular to the light rays) of each point on the echelette surface is plotted against the square of the radial distance (r² or ρ) from the optical axis of the lens (referred to as r-squared space). In multifocal lenses, each echelette 23 may have a diameter or distance from the optical axis which is often proportional to Ain, n being the number of the echelette 23 as counted from optical axis 24. Each echelette has a characteristic optical zone 30 and transition zone 31. Optical zone 30 typically has a shape or downward slope that is parabolic as shown in FIG. 3B. The slope of each echelette in r-squared space (shown in FIG. 3A), however, is the same. As for the typical diffractive multifocal lens, as shown here, all echelettes have the same surface area. The area of echelettes 23 determines the diffractive power of lens 20, and, as area and radii are correlated, the diffractive power is also related to the radii of the echelettes. The physical offset of the trailing edge of each echelette to the leading edge of the adjacent echelette is the step height. An exemplary step height between adjacent echelettes is marked as reference number 33 in FIG. 3A. The step heights remain the same in r-squared space (FIG. 3A) and in linear space (FIG. 3B). The step offset is the height offset of the transition zone from the underlying base curve.

A factor contributing to visual symptoms in diffractive lenses are the transition zones between the echelettes. The transition zones may contribute to adverse visual effects such as halos, glare, and scatter. For example, when light enters the optic surface from an angle, there can be unwanted internal reflection of the light on the surface of a transition zone. Further, the light from different transition zones, originating from different rings on the surface, may interact and cause unwanted diffraction and subsequent constructive interference away from a useful retinal image.

Transition zones may be configured to reduce the possibility of visual symptoms resulting from the transition zones. One or more transition zones may be provided, for example, having at least a portion with an elevated surface roughness. The transition zones may be configured to scatter light that strikes the portion of the transition zone, thus reducing unwanted optical effects. In embodiments, the elevated surface roughness of the transition zone may have a greater roughness than a surface of the optical zone of the echelette.

In embodiments, the roughness of the optical zone of the echelette may be optically smooth, with roughness smaller than λ/(8 cos θ) where λ is the wavelength of the light and θ is the angle of incidence of the light. The elevated surface roughness of the transition zone may be a greater roughness than such a roughness of the optical zone in embodiments.

In embodiments, the surface roughness of the optical zone of the echelette may have a surface roughness resulting from a standard manufacturing process. An elevated surface roughness of the transition zone may be a greater roughness than that of the standard manufacturing process as found on the optical zone of the echelette. The elevated surface roughness may be higher than the roughness of the optical zone. As a result, the scatter originating from the elevated surface roughness may be higher than that of the optical zone of the echelette.

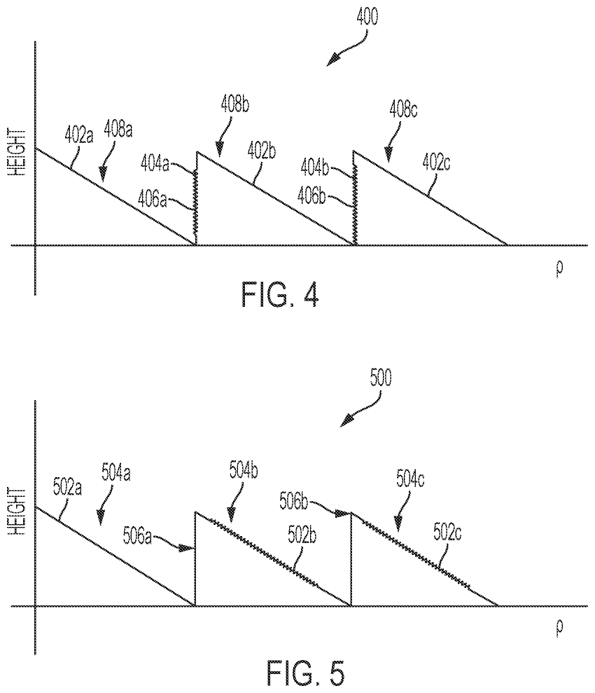

FIG. 4 illustrates such an embodiment of a diffractive profile 400 including a plurality of echelettes 402a—c having transition zones 404a, b and optical zones 408a—c. Each transition zone 404a, b may include a respective portion 406a, b having an elevated surface roughness. In embodiments, only a portion of the transition zone 404a, b may have an elevated surface roughness, or the entirety of the transition zone 404a, b may have the elevated surface roughness. All or a portion of the transition zones of the echelettes of the diffractive profile may include the elevated surface roughness.

The elevated surface roughness may be intentionally created on the transition zones during the manufacturing of the transition zones or during another process that forms the elevated surface roughness. For example, in embodiments, a tool may be utilized to form the elevated surface roughness. The elevated surface roughness may be formed by a tool applied to one or more of the transition zones. The tool in embodiments may comprise a lathe, although in other embodiments other tools such as lasers or other cutting instruments may be utilized. In embodiments, a chemical process may be utilized to form the elevated surface roughness, and in certain embodiments, the elevated surface roughness may be formed by a mold that the optic is formed in. In other embodiments, other methods may be utilized to form the elevated surface roughness.

A tool utilized to form the elevated surface roughness may further be configured to form other portions of the optic, including the transition zones, the optical zones, or other portions including refractive zones as desired. For example, a lathe may cut a portion or the entirety of the optic and may also form the elevated surface roughness.

In embodiments, the elevated surface roughness may be created by forming a diffractive profile with an elevated surface roughness over the entire diffractive surface. The surface of the optic may be formed having the elevated surface roughness. Subsequently, one or more areas of the surface may be polished to reduce the elevated surface roughness of the areas. For example, the process may include polishing areas of one or more of the optical zones to create an optically smooth surface or a smoother surface roughness. Various methods of polishing may be utilized, for example tumble polishing or other forms of polishing. To prevent a transition zone or other portion of the optic from being tumble polished, these zones (or in general, those zones that should keep an elevated surface roughness) may be protected by a cover during tumbling or other polishing process. The portion may be covered during the polishing. Other methods to maintain an elevated surface roughness of one or more transition zones or optical zones (or other portions of the optic) may be utilized as desired.

The elevated surface roughness may comprise a textured pattern on at least a portion of a transition zone. The elevated surface roughness may comprise a frosting on the transition zone that scatters light striking the transition zone.

In the embodiment shown in FIG. 4 the transition zones 404a, b each have an elevated surface roughness that has a greater roughness than a surface of at least one optical zone 408a—c of the plurality of echelettes 402a—c. The transition zones 404a, b each have an elevated surface roughness that has a greater roughness than a surface of all of the optical zones 408a—c of the plurality of echelettes 402a—c of the diffractive profile. The optical zones 408a—c may lack an elevated surface roughness and each may be optically smooth. As such, during a formation process, the optical zones 408a—c may be formed to include optically smooth surfaces. A portion or the entirety of the optical zones 408a—c may be optically smooth surfaces in embodiments. In embodiments, however, the optical zones 408a—c may be formed to include an elevated surface roughness.

FIG. 5, for example, illustrates an embodiment of a diffractive profile 500 including a plurality of echelettes 502a—c in which one or more optical zones 504a—c of the echelettes 502a—c includes an elevated surface roughness. As shown, the optical zones 504b, c of the echelettes 502b, c include an elevated surface roughness, and the optical zone 504a of the echelette 502a is optically smooth. At least one of the optical zones 504a of the plurality of echelettes 502a—c lacks an elevated surface roughness and at least one of the optical zones 504b, c of the plurality of echelettes 502b, c has an elevated surface roughness. At least one of the transition zones 506a, b of the plurality of echelettes 502a—c lacks an elevated surface roughness. The optical zones 504b, c, may each have a greater roughness than a roughness of the transition zones 506a, b. Variations in the number of echelettes that either include an elevated surface roughness or are optically smooth may result. In embodiments, one echelette, a portion of echelettes of a diffractive pattern, or all echelettes of a diffractive pattern, may include an optical zone having an elevated surface roughness.

The elevated surface roughness on the optical zones may be formed in a similar manner as the elevated surface roughness on the transition zones discussed in regard to the embodiment of FIG. 4.

In an embodiment as shown in FIG. 5, the transition zones 506a, b may lack an elevated surface roughness and may be optically smooth.

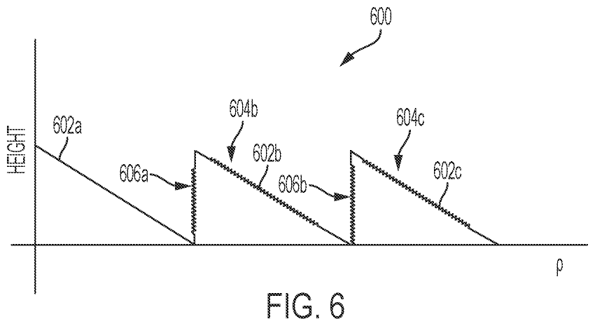

In embodiments, a combination of transition zones and optical zones may include an elevated surface roughness. FIG. 6, for example, illustrates a diffractive profile 600 including a plurality of echelettes 602a—c in which a combination of optical zones 604b, c and transition zones 606a, b include an elevated surface roughness. The portions of the optic to include an elevated surface roughness may be selected and an elevated surface roughness may be applied to the selected portions of the optic as desired. Certain transition zones and/or optical zones (e.g., all or a portion of the transition zones and/or optical zones) may be selected to include an elevated surface roughness as desired. A tool may be utilized to selectively form an elevated surface roughness on all or a portion of the transition zones and/or optical zones, and the elevated surface roughness may be selectively formed on all or a portion of a respective transition zone or optical zone. In embodiments, the elevated surface roughness may be selectively formed on a first one of the transition zones, and an optically smooth second one of the transition zones may be selectively formed. In embodiments, the elevated surface roughness may be selectively formed on a first one of the optical zones, and an optically smooth second one of the optical zones may be selectively formed. Combinations of transition zones and optical zones having elevated surface roughness or being optically smooth may result.

In embodiments, the elevated surface roughness of at least one of the transition zones 606b may extend to at least a portion of the optical zone 604c. The extension may be a continuous elevated surface roughness from the transition zone 606b to the portion of the optical zone 604c, or may be an intermittent elevated surface roughness from the transition zone 606b to the portion of the optical zone 604c. In embodiments, the elevated surface roughness of at least one of the transition zones 606b may extend to one or both of at least a portion of the optical zone 604c of the echelette 602c or at least a portion of an optical zone 604b of an adjacent echelette 602b. The extension may be a continuous elevated surface roughness from the transition zone 606b to the portion of the optical zone 602c and/or the portion of an optical zone 604b of an adjacent echelette 602b, or may be an intermittent elevated surface roughness in embodiments. In embodiments, the elevated surface roughness may extend continuously across one or more echelettes, and may extend over all echelettes of the diffractive profile in embodiments.

In embodiments, one or more of the transition zones 606a, b and/or optical zones 604b, c may have an elevated surface roughness with a greater roughness than a surface of at least one optical zone 602a of the diffractive profile.

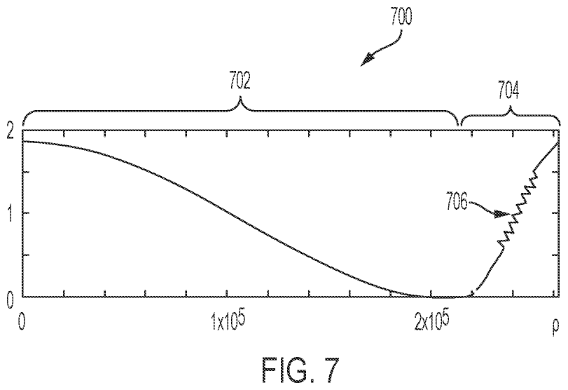

FIG. 7 illustrates an embodiment of a diffractive profile 700 including a single echelette 702, and a transition zone 704 including an elevated surface roughness 706. In embodiments, at least one echelette may comprise the diffractive profile and may have an optical zone and a transition zone 704. At least a portion of the transition zone 704 may have an elevated surface roughness, as disclosed in embodiments herein. At least a portion of the optical surface may include an elevated surface roughness. Other variations in the number of echelettes for the diffractive pattern may be utilized as desired (e.g., at least two, at least three, at least four, at least five echelettes, etc.).

The use of an elevated surface roughness may improve light scatter of light against a surface, thus reducing adverse optical effects for the optic.

An optic for an ophthalmic lens that includes a diffractive profile or refractive profile disclosed herein may be fabricated utilizing a variety of methods. A method may include determining optical aberrations of a patient's eye. Measurements of a patient's eye may be made in a clinical setting, such as by an optometrist, ophthalmologist, or other medical or optical professional. The measurements may be made via manifest refraction, autorefraction, tomography, or a combination of these methods or other measurement methods. The optical aberrations of the patient's eye may be determined.

A determination of the visual range of the patient may also be determined. For example, the ability of the patient to focus on near objects (presbyopia) may be measured and determined. A range of add power for the ophthalmic lens may be determined.

The measurements of the patient's eye may be placed in an ophthalmic lens prescription, which includes features of an optic that are intended to address the optical aberrations of the patient's eye, as well as features that address the visual range for the patient (e.g., an amount of add power and number of focuses to be provided by the optic).

The ophthalmic lens prescription may be utilized to fabricate an optic for the ophthalmic lens. A refractive profile of the optic may be determined based on the ophthalmic lens prescription, to correct for the optical aberrations of the patient's eye. Such a refractive profile may be applied to the optic, whether on a surface including the diffractive profile or on an opposite optical surface. The diffractive profile may also be determined to provide for the desired distribution of add power for the optic.

The determination of one or more of a refractive or diffractive profile and the fabrication of the optic may be performed remotely from the optometrist, ophthalmologist, or other medical or optical professional that performed the measurements of a patient's eye, or may be performed in the same clinical facility of such an individual. If performed remotely, the fabricated optic may be delivered to an optometrist, ophthalmologist, or other medical or optical professional, for being provided to a patient. For an intraocular lens, the fabricated optic may be provided for implant into a patient's eye.

The fabricated optic may be a custom optic fabricated specifically for the patient's eye, or may be fabricated in a manufacturing assembly and then selected by an optometrist, ophthalmologist, or other medical or optical professional for supply to a patient, which may include implantation in the patient's eye.

In embodiments, a user may determine all or a portion of transition zones or optical zones to include an elevated surface roughness. The optic may be fabricated to include the elevated surface roughness on the selected portions.

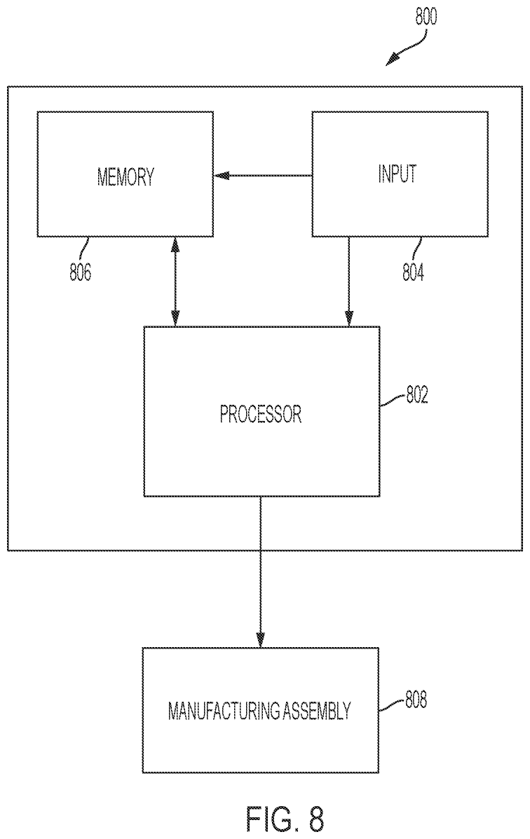

FIG. 8 illustrates an embodiment of a system 800 that may be utilized to perform all or a portion of the methods disclosed herein. The system 800 may include a processor 802, an input 804, and a memory 806. In certain embodiments the system 800 may include a manufacturing assembly 808.

The processor 802 may comprise a central processing unit (CPU) or other form of processor. In certain embodiments the processor 802 may comprise one or more processors. The processor 802 may include one or more processors that are distributed in certain embodiments, for example, the processor 802 may be positioned remote from other components of the system 800 or may be utilized in a cloud computing environment. The memory 806 may comprise a memory that is readable by the processor 802. The memory 806 may store instructions, or features of intraocular lenses, or other parameters that may be utilized by the processor 802 to perform the methods disclosed herein. The memory 806 may comprise a hard disk, read-only memory (ROM), random access memory (RAM) or other form of non-transient medium for storing data. The input 804 may comprise a port, terminal, physical input device, or other form of input. The port or terminal may comprise a physical port or terminal or an electronic port or terminal. The port may comprise a wired or wireless communication device in certain embodiments. The physical input device may comprise a keyboard, touchscreen, keypad, pointer device, or other form of physical input device. The input 804 may be configured to provide an input to the processor 802.

The system 800 may be utilized to perform the methods disclosed herein, such as the processes of determining a diffractive profile of the optic, as well as a refractive profile of the optic. The system 800 may determine whether to include an elevated surface roughness on all or a portion of an optic, such as one or more transition zones or optical zones, or portions of one or more transition zones or optical zones. The processor 802 may be configured to determine the diffractive profile to provide for various add powers for the optic, as well as determining a refractive profile to correct for ocular aberrations of the patient. The processor 802 may be configured to select all or a portion of the optic to form the elevated surface roughness.

The processor 802 may provide the refractive profile and/or diffractive profile to the manufacturing assembly 808, which may be configured to fabricate the optic for the ophthalmic lens based on the refractive profile and/or diffractive profile. The manufacturing assembly 808 may comprise one or more apparatuses for forming the optic, and may comprise a high volume manufacturing assembly or a low volume manufacturing assembly. The manufacturing assembly 808 may be used for manufacture remote to a clinic in which measurements of the individual's eye or made, or local to such a clinic. The manufacturing assembly may include apparatuses such as lathe tools, or other lens formation devices to fabricate the optic. A tool such as a lathe or other manufacturing apparatus may be utilized to form the elevated surface roughness if desired. Other methods may be utilized to form the elevated surface roughness if desired. The manufacturing assembly may be configured to selectively form the elevated surface roughness on one or more of the optical zones or transition zones of the plurality of echelettes. In embodiments, the manufacturing assembly may be configured to form the elevated surface roughness by applying a tool to the portion of the transition zone to cut the elevated surface roughness into the portion.

In embodiments, the manufacturing assembly may be configured to form the elevated surface roughness by forming a surface of the optic having the elevated surface roughness and polishing at least one area of the surface to reduce the elevated surface roughness of the at least one area. The at least one area may include the optical zone of at least one echelette in embodiments. The manufacturing assembly may be configured to form the configurations of optics disclosed herein.

In one embodiment, the processor 802 may be provided with an ophthalmic lens prescription for the individual's eye that may be provided as discussed herein. The processor 802 may receive the ophthalmic lens prescription via the input 804. The processor 802 may determine one or more of the diffractive profile or a refractive profile based on the prescription. The system 800 may fabricate the optic for the ophthalmic lens based on the prescription.

The system 800 may be configured to fabricate any of the embodiments of ophthalmic lenses disclosed herein.

In one embodiment, a diffractive profile as disclosed herein may be positioned on a surface of a lens that is opposite an aspheric surface. The aspheric surface on the opposite side of the lens may be designed to reduce corneal spherical aberration of the patient.

In one embodiment, one or both surfaces of the lens may be aspherical, or include a refractive surface designed to extend the depth of focus, or create multifocality.

In one embodiment, a refractive zone on one or both surfaces of the lens may be utilized that may be the same size or different in size as one of the diffractive zones. The refractive zone includes a refractive surface designed to extend the depth of focus, or create multifocality.

Any of the embodiments of lens profiles discussed herein may be apodized to produce a desired result. The apodization may result in the step heights and step offsets of the echelettes being gradually varied according to the apodization, as to gradually increasing the amount of light in the distance focus as a function of pupil diameter.

The features of the optics disclosed herein may be utilized by themselves, or in combination with refractive profiles of the optics and/or with features providing for correction of chromatic aberrations (e.g., achromats, which may be diffractive).

The ophthalmic lenses disclosed herein in the form of intraocular lenses are not limited to lenses for placement in the individual's capsular bag. For example, the intraocular lenses may comprise those positioned within the anterior chamber of the eye. In certain embodiments the intraocular lenses may comprise “piggy back” lenses or other forms of supplemental intraocular lenses.

Features of embodiments may be modified, substituted, excluded, or combined as desired.

In addition, the methods herein are not limited to the methods specifically described, and may include methods of utilizing the systems and apparatuses disclosed herein.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. An ophthalmic lens comprising:
an optic including a diffractive profile including at least one echelette having an optical zone and a transition zone, with at least a portion of the transition zone having an elevated surface roughness.

2. The ophthalmic lens of claim 1, wherein the elevated surface roughness comprises a frosting on the transition zone.

3. The ophthalmic lens of claim 1, wherein the elevated surface roughness is formed by a tool applied to the transition zone.

4. The ophthalmic lens of claim 3, wherein the tool forms the transition zone.

5. The ophthalmic lens of claim 4, wherein the tool forms the optical zone.

6. The ophthalmic lens of any of claim 5, wherein the tool comprises a lathe.

7. The ophthalmic lens of claim 2, wherein the elevated surface roughness is formed by a mold that the optic is formed in.

8. The ophthalmic lens of any of claim 2, wherein the elevated surface roughness is formed in a process including forming a surface of the optic having the elevated surface roughness and polishing at least one area of the surface to reduce the elevated surface roughness of the at least one area.

9. The ophthalmic lens of claim 8, wherein the at least one area includes the optical zone of the at least one echelette.

10. The ophthalmic lens of any of claim 2, wherein the elevated surface roughness is configured to scatter light striking the transition zone.

11. The ophthalmic lens of any of claim 10, wherein the elevated surface roughness comprises a textured pattern on at least the portion of the transition zone.

12. The ophthalmic lens of any of claim 11, wherein the elevated surface roughness has a greater roughness than a surface of the optical zone.

13. The ophthalmic lens of any of claim 12, wherein the optical zone has a surface that is optically smooth.

14. The ophthalmic lens of any of claim 13, wherein an entirety of the transition zone has the elevated surface roughness.

15. The ophthalmic lens of any of claim 11, wherein the elevated surface roughness extends to at least a portion of the optical zone.

16. The ophthalmic lens of any of claim 1, wherein the diffractive profile includes a plurality of echelettes, each echelette having an optical zone and a transition zone.

17. The ophthalmic lens of claim 16, wherein the elevated surface roughness has a greater roughness than a surface of at least one optical zone of the plurality of echelettes.

18. The ophthalmic lens of claim 17, wherein at least one of the transition zones of the plurality of echelettes lacks an elevated surface roughness.

19. The ophthalmic lens of any of claim 18, wherein at least one of the optical zones of the plurality of echelettes lacks an elevated surface roughness and at least one of the optical zones of the plurality of echelettes has an elevated surface roughness.

20. The ophthalmic lens of any of claim 19, wherein the elevated surface roughness of at least the portion of the transition zone extends to one or both of at least a portion of the optical zone of the at least one echelette or at least a portion of an optical zone of an adjacent echelette.

21-47. (canceled)
